(19) United States
(12) Patent Application Publication
(10) Pub. No.: US 2021/0358641 A1
(43) Pub. Date: Nov. 18, 2021

(54) BLOCKCHAIN-BASED HEALTH MONITORING SYSTEM

(71) Applicant: ALI GROUP S.R.L. - CARPIGIANI, Cernusco Sul Naviglio (Milano) (IT)

(72) Inventors: Andrea Cocchi, Calderara di Reno (Bologna) (IT); Carlo Giannelli, Bologna (IT); Roberto Lazzarini, Reggio Emilia (IT); Cesare Stefanelli, Bologna (IT); Federico Tassi, Bologna (IT)

(21) Appl. No.: 17/306,339

(22) Filed: May 3, 2021

(30) Foreign Application Priority Data
May 13, 2020 (IT) 102020000010861

(51) Int. Cl. G16H 50/80; G06F 16/23; G06K 7/14; G16H 10/40; G16H 10/65; H04L 9/06; G01N 35/00

(52) U.S. Cl. CPC G16H 50/80; G06F 16/2379; G06K 7/1413; G16H 10/40; G01N 2035/00881; H04L 9/0643; G01N 35/00871; H04L 2209/38; G16H 10/65

(57) ABSTRACT

A health supervision system, including: an identification station, configured to obtain at least one first data item indicating an identity of a user, a health station configured to obtain at least one second data item indicating a health situation of the user identified, at least one local data repository, and a control unit, configured for receiving the at least one first data item and the at least one second data item, calculating a string, using a cryptographic function, as a function of the at least one first data item and/or the at least one second data item, storing the at least one first data item and/or the at least one second data item in the at least one local data repository, and transmitting the string to a distributed architecture database of the distributed ledger type to write the string to the distributed architecture database.

This application claims priority to Italian Patent Application 102020000010861 filed May 13, 2020, the entirety of which is incorporated by reference herein.

### BACKGROUND OF THE INVENTION

This invention relates to a health supervision system, for example, a health supervision system capable of recording the health situations of a plurality of users.

The spread of Covid-19 has led to the adoption of restrictive measures which have resulted in a drastic reduction in trade and which, in many cases, have brought production activities to a halt. Concluding this period of restrictions will necessarily require highly precautionary measures to prevent new clusters of infection. Until such time as vaccines become available, the risk not only of the resurgence of internal clusters but also of importing infections from abroad remains high. In this context, work places are considered potential sources of biological hazard.

In the current context, therefore, the many new challenges that are emerging involve minimizing and monitoring new infections or improving the safety of people at work.

Monitoring the state of health of users may be linked to one or more of the following issues: ensuring data immutability, allowing access to information only by authorized persons (for example, health and/or judicial authorities) and guaranteeing the privacy and the right to be forgotten of the persons monitored.

### SUMMARY OF THE INVENTION

This invention therefore has for an aim to meet the above mentioned needs by providing a health supervision system as described in this disclosure, capable of monitoring the health situation of the public with a view to limiting the spread of the virus.

Also an aim of this invention is to allow recorded data to remain immutable while at the same time guaranteeing the users' right to be forgotten.

A further aim of this invention is to ensure the traceability of the tests performed, as set out in Resolution No. 350, dated Apr. 16, 2020, passed by the Emilia Romagna regional council.

### BRIEF DESCRIPTION OF THE DRAWINGS

The technical features of the disclosure, with reference to the above aims, are clearly described in the claims below and its advantages are apparent from the detailed description which follows, with reference to the accompanying drawings which illustrate a preferred, non-limiting example embodiment, and in which:

FIG. 1 shows a block diagram representing a health supervision system according to one or more embodiments.

### DESCRIPTION OF THE PREFERRED EMBODIMENTS

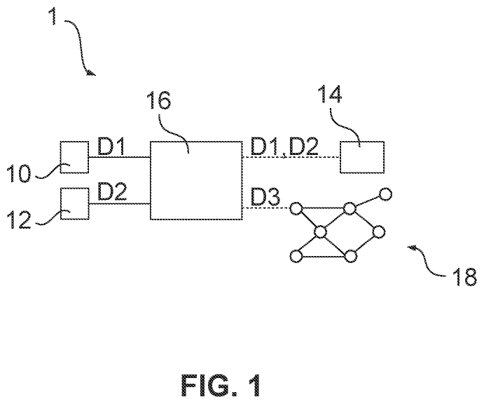

FIG. 1 shows a health supervision system 1 according to one or more embodiments. The system 1 allows setting up a company check-point where users, for example, employees, visitors and members of their families, can undergo epidemiological screening on a voluntary basis. Test results, for example, in the form of second data items D2, can be recorded in the local data repository 16, for example, a company database, and can be certified through blockchain technology as described in more detail below. Advantageously, it is thus possible to periodically monitor the state of health of a group of users in order to provide better protection for the company. For example, the user can periodically interact with the system 1 to undergo the health check-up. For example, each user can interact with the system 1 at predetermined regular intervals, for example, weekly or twice weekly.

Advantageously, converting a company into a health center to guarantee the health of employees and members of their families can make it easier for shops and restaurants and other trading enterprises to reopen safely, thus allowing a safe and effective transition out of the emergency situation.

The system 1 comprises:
an identification station 10, configured to obtain at least one first data item D1 indicating an identity of a user,
a health station 12 configured to obtain at least one second data item D2 indicating a health situation of the user identified,
at least one local data repository 14, and
a control unit 16, configured for:
   receiving the at least one first data item D1 and the at least one second data item D2,
   calculating a string D3, as a function of the at least one first data item D1 and/or of the at least one second data item D2, using a cryptographic function, where the string D3 indicates, for example, the first data item D1 and/or the second data item D2,
   storing the at least one first data item D1 and/or the at least one second data item D2 in the at least one local data repository 14, and
   transmitting the string D3 to a distributed architecture database 18 of the distributed ledger type to write the string D3 to the distributed architecture database, for example, a blockchain-based database.

According to an aspect, the system 1 may comprise the distributed architecture database 18 of the distributed ledger type. For example, the database 18 may be defined by a peer-to-peer network based on blockchain technology. The database 18 may be defined by a plurality of nodes within one or more companies forming part of a health supervision network, and/or within local health care authorities (ASL).

According to an aspect, the control unit 16 may be configured to write the string D3 to the distributed architecture database 18.

Advantageously, this system allows ensuring the immutability of the data, in that it is based on the use of the distributed architecture database 18, while at the same time guaranteeing the users' right to be forgotten, in that the at least one first data item D1 and the at least one second data item D2 are not stored in the database 18. Instead, the string D3, indicating the data items D1 and D2, is stored in the database 18. In effect, if the results of an employee's swab test were stored in a blockchain, that information would become immutable (tamper proof) but would be visible to one or more organizations forming part of the same blockchain, in contrast with the right to personal data privacy, whose observance is even more stringent in the case of sensitive or health data.

In one or more embodiments, the identification station 10 may comprise a scanner, preferably a bar code scanner or a camera, configured to obtain the at least one first data item D1 comprising one or more elements identifying the user's identity. For example, the scanner may be configured to identify a user by means of an identification document such as, for example, an identity card, driver's license and/or social security card. The scanner may thus capture the at least one first data item D1, which may include the name, surname and/or taxpayer ID number of a user. The identification station 10 may be connected directly or indirectly to the control unit 16 and may be configured to transmit the at least one first data item D1 to the control unit 16.

In one or more embodiments, the health station 12 may comprise a laboratory configured to perform a serological and/or molecular test on a biological sample extracted (taken) from the user identified and to obtain the at least one second data item D2 including a result of the serological and/or molecular test. The health station 12 may be connected directly or indirectly to the control unit 16 and may be configured to transmit the at least one second data item D2 to the control unit 16.

According to an aspect, the health supervision system 1 may comprise the offices of one or more companies. The identification station 10 may be located inside the offices of the one or more companies, each company having, associated with it, an identification station 10 and a health station 12. The health station 12—for example, the laboratory—may be situated inside the offices of the one or more companies or it may be situated at a remote location, off company premises. In the latter case, after identification, the sample extracted (taken) from the user can be transmitted to the health station 12 for processing.

According to an aspect, the laboratory comprises highly qualified staff capable of working with the companies based on industrial methods and schedules.

According to an aspect, the health station 12 may be configured to perform a rapid serological test by analyzing a blood sample using immuno-chromatography plates to detect IG-G and IG-M antibodies. The health station 12 may comprise a plurality of disposable kits capable of performing the serological test and providing a result in a relatively short space of time.

The epidemiological screening process might require that each person involved undergo a rapid serological test (subject to consent). In the event of a negative result (IG-M and IG-G negative), the test may be repeated within a predetermined time interval (for example, 15-20 days). If the rapid serological test gives a positive result (IG-M and/or IG-G), the screening process may involve a chemiluminescence analysis or ELISA. After the chemiluminescence test or ELISA, the screening process may comprise performing a molecular test to confirm the diagnosis for those who tested IG-M positive and IG-G positive or negative.

The chemiluminescence test or ELISA is a semi-quantitative in vitro assay for human antibodies, constituting cogent evidence to integrate the direct search for the pathogen. The instruments required include the specific approved kits as well as accessory instruments useful for performing the test. To obtain the result of the ELISA test, the laboratory may comprise basic laboratory instruments and consumables (calibrated pipettes, tips, chronometer, incubator and thermal bath, refrigerators) as well as instruments for reading the result (spectrophotometer).

The health station 12 may also be configured to carry out a molecular test based on RT-PCR methods to amplify the viral genes expressed during infection by SARS-CoV-2. In this case, the laboratory may also comprise instruments such as centrifuge, tweezers, a detector for nucleic acid (DNA/RNA) sample quality analysis and analysis of genes of pathogens derived from the human body by single fluorescence PCR.

The health station 12 may be divided into dedicated areas: for example, a first area for taking the sample; a second area for collecting the samples, in the case where the samples are processed off company premises; and lastly, a third laboratory area for analysis and final checking. If the health station 12 is totally in-house, the first, second and third areas may all be situated on company premises; alternatively, the third area, comprising the laboratory, may be situated at a location outside the company.

Advantageously, creating an in-house laboratory would allow reducing test times, through centralization of structures, attaining a high degree of innovation compared to the state of the art, with benefits for the entire production chain. Compared to using an external laboratory, an in-house laboratory would also be advantageous in economic and financial terms in the long term and considering the large number of tests to be carried out to keep employees and their families monitored.

According to an aspect, the system 1 may require consent to process the data collected, that is, to process and record the at least one first data item D1 and the at least one second data item D2, whether the result is positive or negative. For example, storage may occur through use of a certified data repository.

As stated above, the system 1 can be subdivided into a network of companies, so as to be able to monitor a plurality of users. Each company can therefore record the first and second data items D1, D2 independently of each other, for example, in one or more local data repositories 14 and each company can record the data strings D3 in the same distributed architecture database 18 based on the same blockchain, for example, to ensure that all the data are recorded correctly and immutably.

Advantageously, when a relatively large number of companies forms part of the same blockchain, the health supervision system 1 can become tamper proof, or almost tamper proof, for example, even without the collaboration of local health authorities (ASL) or other public authorities.

According to an aspect, the at least one data repository 14 may be connected to the control unit 16, in hardwired or wireless manner, and may be configured to receive from it the at least one first and/or second data item D1, D2.

In one or more embodiments, the at least one local data repository 14 comprises a centralized data repository, the data repository being protected and accessible to the control unit 16 subject to authorization.

For example, the first data items D1 and the second data items D2 may be accessed only through the control unit 16, for example, after entering a password. That way, access to the sensitive data is possible only if authorized.

For example, the first and second data items D1, D2 may be accessible only to public health and/or judicial organizations or to a company doctor.

In addition, or alternatively, the at least one local data repository 16 may be defined at least partly by a plurality of personal electronic devices, preferably personal smartphones. For example, a certain electronic device may be associated with each user. That way, the at least one second data item D2 of a certain identified user can be saved only to the personal electronic device of that user. In other words, the second data items D2 of individual users can be saved to different personal devices (a copy of the data items may be present in a centralized company database).

In addition, or alternatively, the at least one second data item D2 is accessible following a request from the user and/or from the control unit 16. Advantageously, the user can access their personal data even outside the context of the company, for example, if they are required to show they are negative to the virus in other circumstances, such as at a cinema, restaurant, stadium, and so on. At the same time, the control unit 16 can access the data present in the personal electronic devices, so that public health and/or judicial organizations can obtain the first and second data items D1, D2 of each user in the event of need.

In addition, or alternatively, the at least one second data item D2 can only be deleted following a command from the control unit 16. Advantageously, that way, the user cannot delete the second data items D2.

The second data items D2 in the personal electronic devices may be accessible to the user through a mobile application, hereinafter called “app”.

The use of an app containing the results of tests may have one or more advantages: for example, the user can show the test results through the app in order to prove the absence of infection at the date of the test, so as to allow public commercial establishments to interact with their customers in a safe environment.

That way, at least part of the local data repository 14 might be resident in personal electronic devices.

According to an aspect, the database 18 may be connected to the control unit 16, in hardwired or wireless manner, and may be configured to receive from it the string D3.

In one or more embodiments, the distributed architecture database 18 of distributed ledger type is of the blockchain type and comprises information grouped in a plurality of information blocks interconnected by cryptographic algorithms.

According to an aspect, creating a blockchain shared by different companies in a certain geographical area may have one or more advantages: for example, it allows certifying the immutability of the first and second data items D1, D2 recorded by each individual company. For example, every company may record the data D1, D2 independently of the others in local data repositories 14 and, at the same, may record strings D3, indicating the data D1, D2 in the same blockchain, so as to ensure that all the data are recorded correctly and immutably.

Advantageously, when a sufficiently large number of different companies record their data on the blockchain, it will be easier to guarantee a tamper-proof system.

According to an aspect, the distributed architecture database 18 of the distributed ledger type may be defined by or built on the Ethereum or Ethereum Classic or Iota or Eos or NEO or Waves or Qtum or NEM or Multiversum or R3 Corda or R3 Corda enterprise or Hyperledger or Ripple or Stellar platform.

According to an aspect, the distributed architecture database 18 of the distributed ledger type may operate with a consensus protocol for writing to the database 18 and selected from the following types:
proof of work;
proof of stake;
Corda consensus protocols (configured to reach consensus on the specific “state object”);
CFT (Crash fault tolerant), preferably implemented with Kafka and/or Zookeeper;
Solo;
BFT (Byzantine fault tolerance);
PBFT (practical BFT);
SBFT (simplified BFT);
Raft;
Sumeragi;
PoET (proof of Elapsed Time);
Permissioned Voting-based.

As stated earlier herein, the database 18 may be defined by a plurality of processing nodes. Each company may be associated with a specific node which is the first to check and consent to the addition of strings D3 to the distributed architecture database 18 of the distributed ledger type.

According to an aspect, the processing nodes may comprise a first set of processors which are configured to keep a complete copy of the distributed architecture database 18 and a second set of processors which are configured to keep a partial copy of the distributed architecture database 18. In this way, advantageously, the processing nodes of the second set can be simpler than those of the first set and integrate a reduced capacity memory.

Preferably, each string D3 has a time stamp associated with it. Preferably, the strings D3 may be interconnected with each other by cryptographic algorithms.

In one or more embodiments, the control unit 16 is therefore configured to record, retrieve and/or delete the first and second data items D1, D2 in the at least one local data repository 14. At the same time, the control unit 16 is configured to save the string D3 indicating the first and/or second data items D1, D2 to the database 18.

The string D3 may indicate the data items D1, D2 but at the same time it can protect the user's privacy in that the data items D1, D2 are not visible in unencrypted form. In other words, the string D3 can be calculated cryptographically.

The step of calculating the string with a cryptographic function may comprise calculating the string D3 with a cryptographic hash function, for example, with a secure hash algorithm SHA-256.

In one or more embodiments:
the calculated string D3 has a biunique match with the at least one first data item D1 and/or the at least one second data item D2 from which the string D3 is calculated, and/or
the at least one first and/or second data item D1, D2 cannot be obtained from the string D3.

That way, the test results—that is, the unencrypted second health data items D2—can be saved in the local data repository 14, where the accessibility of the data repository 14 is subject to authorization. On the contrary, the strings D3, indicating at least the second data items D2 from which they are calculated, are freely accessible by anyone who has access to the database 18 so as to allow the data to be certified. In effect, the truthfulness of the data provided is guaranteed by the fact that the secure hash of these results has been saved in the blockchain.

In one or more embodiments, the control unit 16 is configured for:
associating a time stamp with the at least one first data item D1 and/or with the at least one second data item D2, and
deleting data—automatically, for example—between a plurality of first data items D1 and/or second data items D2 stored in the at least one local data repository 14 if the time stamp indicates that the data were stored before a certain date—for example, if the data were saved on or before a date six months previously.

Advantageously, this allows protecting the privacy and the right to be forgotten of users subjected to health supervision, in compliance with Italian and European regulations and with the recommendations of the data protection authority. In effect, the data recorded are not published in unencrypted form in the database 18 and can be deleted after a predetermined time interval which is, for example, established in agreement with the authorities. Upon the lapse of that time period, the system 1 automatically removes the obsolete data.

In one or more embodiments, the control unit 16 may be configured to transmit an alarm signal if the at least one second data item D2 indicates the presence of SARS-CoV-2 virus antigens, that is to say, if the user might be a carrier of serious acute respiratory syndrome coronavirus 2 or might exhibit symptoms of Covid-19.

According to an aspect, the alarm may be displayed on the personal electronic device of the user who has tested positive to the virus; in addition, or alternatively, the alarm may be transmitted to a company officer, for example, the doctor, to allow starting a procedure to contain the spread of infection.

One or more embodiments address a health supervision method, comprising the following steps:
providing a health supervision system 1 according to one or more embodiments,
obtaining with the identification station 10 at least one first data item D1 indicating the identity of a user,
obtaining with the health station 12 at least one second data item D2 indicating the health situation of the user identified,
receiving with the identification station 10 and the health station 12 the at least one first data item D1 and the at least one second data item D2,
calculating a string D3, using a cryptographic function, as a function of the at least one first data item D1 and/or the at least one second data item D2,
storing the at least one first data item D1 and/or the at least one second data item D2 in the at least one local data repository 14, and
transmitting the string D3 to a distributed architecture database 18 of the distributed ledger type to write the string D3 to the distributed architecture database 18.

According to an aspect, the supervision method may comprise writing the string D3 to the database 18.

According to another aspect, the method can comprise the following steps:
identifying a user;
reading the distributed architecture database 18 and the local data repository 14 to retrieve information associated with the user;
issuing an alarm signal if the reading of the distributed architecture database 18 in combination with the local data repository 14 gives as its result an anomalous health condition associated with the user.

What is claimed is:

1. A health supervision system, comprising:
an identification station, configured to obtain at least one first data item indicating an identity of a user,
a health station configured to obtain at least one second data item indicating a health situation of the user identified,
at least one local data repository, and
a control unit, configured for:
receiving the at least one first data item and the at least one second data item,
calculating a string, using a cryptographic function, as a function of the at least one first data item and/or the at least one second data item,
storing the at least one first data item and/or the at least one second data item in the at least one local data repository, and
transmitting the string to a distributed architecture database of the distributed ledger type to write the string to the distributed architecture database.

2. The health supervision system according to claim 1, wherein the identification station comprises a scanner, preferably a camera or a bar code scanner, configured to obtain the at least one first data item comprising one or more elements identifying the user's identity.

3. The health supervision system according to claim 1, wherein the health station comprises a laboratory configured to perform a serological and/or molecular test on a sample from the user and to obtain the at least one second data item comprising a result of the serological and/or molecular test.

4. The health supervision system according to claim 1, wherein the at least one local data repository comprises a centralized data repository, the data repository being protected and accessible to the control unit following an authorization.

5. The health supervision system according to claim 1, wherein the at least local data repository is defined at least partly by a plurality of personal electronic devices, preferably smartphones, where each of the personal electronic devices is associated with a certain user, so that:
the at least one second data item can be stored only in the personal electronic device of the user identified, and/or
the at least one second data item is accessible following a request from the user and/or from the control unit, and/or
the at least one second data item can only be deleted following a command from the control unit.

6. The health supervision system according to claim 1, comprising the distributed architecture database of distributed ledger type.

7. The health supervision system according to claim 6, wherein the distributed architecture database of distributed ledger type is of the blockchain type and comprises information grouped in a plurality of information blocks interconnected by cryptographic algorithms.

8. The health supervision system according to claim 6, wherein the distributed architecture database of the distributed ledger type is built on the Ethereum or Ethereum Classic or Iota or Eos or NEO or Waves or Qtum or NEM or Multiversum or R3 Corda or R3 Corda enterprise or Hyperledger or Ripple or Stellar platform.

9. The health supervision system according to claim 1, wherein the distributed architecture database of the distributed ledger type operates with a consensus protocol for writing to the database and selected from the following types:
proof of work;
proof of stake;
Corda consensus protocols (configured to reach consensus on the specific “state object”);
CFT (Crash fault tolerant), preferably implemented with Kafka and/or Zookeeper;
Solo;
BFT (Byzantine fault tolerance);
PBFT (practical BFT);
SBFT (simplified BFT);
Raft;
Sumeragi;
PoET (proof of Elapsed Time);
Permissioned Voting-based.

10. The health supervision system according to claim 1, wherein the step of calculating the string comprises calculating the string using a cryptographic hash function.

11. The health supervision system according to claim 1, wherein:
the string is biuniquely matched with the at least one first data item and/or the at least one second data item from which the string is calculated, and/or
the at least one first data item and/or the at least one second data item cannot be obtained from the string.

12. The health supervision system according to claim 1, wherein the control unit is configured for:
associating a time stamp with the at least one first data item and/or with the at least one second data item, and
deleting data between a plurality of first data items and/or second data items stored in the at least one local data repository if the time stamp indicates that the data was stored before a certain date.

13. The health supervision system according to claim 1, wherein the control unit is configured for transmitting an alarm signal if the at least one second data item indicates the presence of SARS-CoV-2 virus antigens.

14. A health supervision method, comprising:
providing a health supervision system according to claim 1,
obtaining with the identification station at least one first data item indicating the identity of a user,
obtaining with the health station at least one second data item indicating the health situation of the user identified,
receiving with the identification station and the health station the at least one first data item and the at least one second data item,
calculating a string, using a cryptographic function, as a function of the at least one first data item and/or the at least one second data item,
storing the at least one first data item and/or the at least one second data item in the at least one local data repository, and
transmitting the string to a distributed architecture database of the distributed ledger type to write the string to the distributed architecture database.
